(19) United States
(12) Patent Application Publication
(10) Pub. No.: US 2024/0066225 A1
(43) Pub. Date: Feb. 29, 2024

(54) Arrangement for a Drug Delivery Device and Drug Delivery Device

(71) Applicant: Sanofi, Paris (FR)

(72) Inventors: Tomas Correa, Hertforshire (GB); Uwe Dasbach, Frankfurt am Main (DE); Thomas Mark Kemp, Hertfordshire (GB); Jeffrey Morang, Bridgewater, NJ (US); Alex Zuyev, Bridgewater, NJ (US)

(21) Appl. No.: 18/253,303

(22) PCT Filed: Nov. 18, 2021
(86) PCT No.: PCT/EP2021/082072

§ 371 Date: May 17, 2023

(30) Foreign Application Priority Data
Nov. 18, 2020 (EP) 20315456.2

(51) Int. Cl. A61M 5/20; A61M 5/32

(52) U.S. Cl. CPC A61M 5/20; A61M 5/3204; A61M 2005/2013; A61M 2005/2026; A61M 2005/206; A61M 2005/208; A61M 2205/19

(57) ABSTRACT

An arrangement for a drug delivery device includes a housing with a proximal end and a distal end, a needle shield remover configured to be rotationally locked to a needle shield covering a needle, and a guiding interface. The needle shield remover is releasably connectable to the housing. The guiding interface is configured such that, when the needle shield remover rotates with respect to the housing from a state in which the needle shield remover is connected to the housing, the guiding interface guides a movement of the needle shield remover relative to the housing in a direction away from the proximal end of the housing to disconnect the needle shield remover from the housing. Movement of the needle shield remover relative to the housing to disconnect the needle shield remover includes an axial movement and a rotational movement of the needle shield remover relative to the housing.

### CROSS REFERENCE TO RELATED APPLICATIONS

The present application is the national stage entry of International Patent Application No. PCT/EP2021/082072, filed on Nov. 18, 2021, and claims priority to Application No. EP 20315456.2, filed on Nov. 18, 2020, the disclosures of which are incorporated herein by reference.

### TECHNICAL FIELD

The present disclosure relates to an arrangement, e.g. an arrangement for a drug delivery device, with a needle shield remover. The present disclosure further relates to a drug delivery device comprising the arrangement.

### BACKGROUND

Drug delivery devices, such as auto-injectors, are known in the art for dispensing a medicament to the injection site of a patient. Such drug delivery devices typically comprise a housing and a cap. A needle syringe is located in the housing and is covered by a needle shield. The cap and needle shield are removable attached to the housing to shield the needle of the needle syringe. To dispense the medicament, the cap and needle shield are first removed from the housing to expose the needle. The needle is then inserted into the body of the patient at the injection site to inject the medicament.

The force required to remove the cap from the housing can be relatively high which can be due to the frictional interface between the needle shield and the syringe. Infirm patients such as the elderly or physically impaired may find removing the cap difficult due to the relatively high forces required. Furthermore the force required to remove the cap can be increased further by cold temperatures, as some medicaments and therefore some drug delivery devices need to be stored in the fridge at low temperatures. The cold temperature may cause the materials to shrink, which leads to a narrowing of the fits between different parts. Also, increasing shelf time may increase the stiction between the needle shield and the syringe. This can exacerbate the difficulty in removing the cap from the housing.

### SUMMARY

The present disclosure provides an improved arrangement for a drug delivery device and an improved drug delivery device. Advantageous embodiments and refinements are subject to the disclosure below.

In an embodiment there is provided an arrangement for a drug delivery device comprising a housing with a proximal end and a distal end, a needle shield remover configured to be rotationally locked to a needle shield covering a needle, wherein the needle shield remover is releasably connected or releasably connectable to the housing, wherein the arrangement comprises a guiding interface, wherein the guiding interface is configured such that, when the needle shield remover rotates with respect to the housing from a state in which the needle shield remover is connected to the housing, the guiding interface guides movement of the needle shield remover relative to the housing in a direction away from the proximal end of the housing to disconnect the needle shield remover from the housing, and wherein the movement of the needle shield remover relative to the housing to disconnect the needle shield remover from the housing comprises axial and rotational movement of the needle shield remover relative to the housing.

As the needle shield remover rotates when being disconnected from the housing and the needle shield remover is rotationally locked to the needle shield, the force driving the rotation may be used to detach the needle shield from the needle or the force may be used to rotate the needle shield with the needle and with needle shield remover to avoid relative rotation of the needle shield relative to the needle. The cooperation between the cap and the housing via the guiding interface can considerably reduce the force required to remove the cap from the housing.

In an embodiment the movement of the needle shield remover relative to the housing comprises a simultaneous axial and rotational movement of the needle shield remover relative to the housing.

In an embodiment the needle shield remover is a cap or part of a cap assembly which comprises a cap and the needle shield remover, wherein, in the cap assembly, the needle shield remover is assembled to the cap, and wherein, in the cap assembly, the needle shield remover is rotationally locked to the cap, e.g. with respect to a surface of the cap, e.g. to an exterior surface of the cap. The exterior surface may be provided as an interface surface for being touched by a user when removing the cap from the housing.

The needle shield remover may be received in a sleeve-like portion of the cap. The needle shield remover may be rotationally and axially locked relative to the cap.

This concept requires the cap and the needle shield remover to rotate together, and thereby allows the functionality of the cap and the needle shield remover to be combined in a single component. Due to the rotational locking of the needle shield remover relative to the cap, a rotation of the cap immediately leads to a rotation of the needle shield remover. This ensures direct power transfer from the cap to the needle shield remover.

In an embodiment, the guiding interface is formed between the cap and the housing.

In an embodiment the guiding interface defines a rotation direction for the rotation required to disconnect the needle shield remover from the housing.

In an embodiment the guiding interface is configured such that rotation in only one direction is permitted to disconnect the needle shield remover from the housing.

In an embodiment the guiding interface comprises at least one guide track which is configured to guide the movement of the needle shield remover relative to the housing when the needle shield remover is disconnected from the housing.

In an embodiment the guide track may be arranged on a part of the cap.

In an embodiment the guide track has at least one sloped region.

Via the slope of the guide track, it is possible to generate a force transmission that makes it easier to remove the cap with the needle shield from the housing.

In an embodiment the guide track comprises differently sloped regions.

Due to differently sloped regions of the guide track, the slope of the guide track can be adjusted to the force curve of the force required to remove the needle shield from the housing.

In an embodiment the guiding interface comprises at least one interaction feature.

In an embodiment the interaction feature is arranged on a part of the housing.

In an embodiment the guiding interface is established between the cap and the housing, and wherein the interaction feature is be located on the housing and the guide track is located on the cap or vice versa.

In an embodiment the arrangement comprises a further guiding interface which is configured to guide movement of the needle shield remover relative to the housing during disconnection of the needle shield remover from the housing, and wherein a rotational direction defined by the further guiding interface is opposite to the one defined by the guiding interface.

Thus, the user can take advantage of the guiding interface regardless of the direction in which he rotates the cap relative to the housing. This additionally simplifies the use of the arrangement.

In an embodiment the housing is configured to receive a reservoir or the reservoir is received in the housing, the reservoir comprising a reservoir body with a discharge opening, an interior which is configured to retain a medicament, and the needle for providing fluid communication between the interior of the reservoir body and the exterior of the reservoir body, and wherein the needle is covered by the needle shield. The reservoir may be a pre-filled syringe. The reservoir body may be of glass.

In an embodiment the reservoir, when received in the housing, is axially secured relative to the housing, wherein the axial position of the reservoir relative to the housing does not change when the needle shield remover is being disconnected from the housing. Preferably, the reservoir is axially fixed relative to the housing after removal of the cap and no axial movement of the reservoir relative to the housing is possible.

In an embodiment the arrangement comprises reservoir biasing system, wherein the reservoir biasing system biases the reservoir with a force in the distal direction relative to the housing during removal of the cap from the housing.

In an embodiment, the force applied by the reservoir biasing system is constant throughout the removal of the cap.

In an embodiment, the force applied by the reservoir biasing system varies throughout the removal of the cap.

In an embodiment, the force applied by the reservoir biasing system decreases throughout the removal of the cap.

In an embodiment, the force applied by the reservoir biasing system increases throughout the removal of the cap.

In an embodiment the reservoir is configured such that, when the needle shield remover is being disconnected from the housing, the reservoir can rotate together with the needle shield remover relative to the housing from an initial reservoir position relative to the housing to an end reservoir position relative to the housing.

The joint rotation of the reservoir together with the needle shield remover relative to the housing can prevent the needle shield from twisting. Torsion of the needle shield is particularly disadvantageous because it can increase the force required to remove the needle shield from the reservoir. Furthermore, torsion of the needle shield may damage the connection of the needle to the reservoir body.

In an embodiment the needle shield remover is configured such that, when the needle shield remover is disconnected from the housing, the needle shield remover rotates from an initial needle shield remover position relative to the housing to an end needle shield remover position relative to the housing, wherein a rotation angle of the reservoir between the initial reservoir position and the end reservoir position is a first angle, wherein a rotation angle of the needle shield remover between the initial needle shield remover position and the end needle shield remover position is a second angle, and wherein the first angle is equal to the second angle.

As mention above, a joint rotation of the reservoir together with the needle shield remover relative to the housing prevents the needle shield from twisting.

In an embodiment the needle shield remover is configured such that, when the needle shield remover is disconnected from the housing, the needle shield remover rotates from an initial needle shield remover position relative to the housing to an end needle shield remover position relative to the housing, wherein a rotation angle of the reservoir between the initial reservoir position and the end reservoir position is a first angle, wherein a rotation angle of the needle shield remover between the initial needle shield remover position and the end needle shield remover position is a second angle, and wherein the first angle is less than the second angle.

As mentioned above, a joint rotation of the reservoir together with the needle shield remover relative to the housing may prevent the needle shield from twisting relative to the reservoir body. However, it may be sufficient if the reservoir does not take part in the entire rotational movement of the needle shield remover, but only during a part of the rotational movement, e.g. that part which requires the greatest force. For achieving this, the rotation of the reservoir relative to the housing during the disconnection of the needle shield remover from the housing may be blocked by a rotational stop, before the needle shield remover has reached its rotational end position relative to the housing.

In an embodiment the first angle is greater than or equal to 15°, 30°, 45°, 60°, 75°, 90°, 105°, 120°, 135°, 150°, 165°, 180°, 195°, 210°, 225°, 240°, 255°, 270°, 285°, 300°, 315°, 330°, 345° or 360°.

In an embodiment the second angle is greater than or equal to 15°, 30°, 45°, 60°, 75°, 90°, 105°, 120°, 135°, 150°, 165°, 180°, 195°, 210°, 225°, 240°, 255°, 270°, 285°, 300°, 315°, 330°, 345° or 360°.

In an embodiment the first angle is less than or equal to 15°, 30°, 45°, 60°, 75°, 90°, 105°, 120°, 135°, 150°, 165°, 180°, 195°, 210°, 225°, 240°, 255°, 270°, 285°, 300°, 315°, 330°, 345° or 360°.

In an embodiment the second angle is less than or equal to 15°, 30°, 45°, 60°, 75°, 90°, 105°, 120°, 135°, 150°, 165°, 180°, 195°, 210°, 225°, 240°, 255°, 270°, 285°, 300°, 315°, 330°, 345° or 360°.

In an embodiment the first angle is between 15° and 360°.

In an embodiment the second angle is between 15° and 360°.

In an embodiment the arrangement further comprises a plunger rod and an energy storage member having energy stored therein, which energy, when released, drives the plunger rod in the distal direction relative to the housing.

In an embodiment there is provided a drug delivery device, which comprises the arrangement, preferably as described further above.

In an embodiment the drug delivery device is an autoinjector.

In an embodiment the drug delivery device is a disposable device.

In an embodiment the drug delivery device is a fixed dose device.

In an embodiment the drug delivery device is a fixed dose device, capable of delivering multiple individual predetermined doses.

In an embodiment the drug delivery device has an elongated shape relative to a bearing surface on the body of a user.

In an embodiment the drug delivery device comprises the reservoir filled with drug.

In an embodiment the drug delivery device comprises a needle shroud, the needle shroud being axially movable relative to the housing.

In an embodiment the dispensing of a drug is triggered by an axial movement of the needle shroud relative to the housing.

These and other aspects, advantages and expediencies will become apparent from the embodiments described hereinafter.

### BRIEF DESCRIPTION OF THE FIGURES

Embodiments will now be described, by way of example only, with reference to the accompanying drawings, in which:

FIG. 1 shows a section of an axial section view of an embodiment;

FIG. 2 shows another section of an axial section view of the embodiment of FIG. 1; and

FIG. 3 shows yet another section of a section view of the embodiment of FIG. 1;

### DETAILED DESCRIPTION

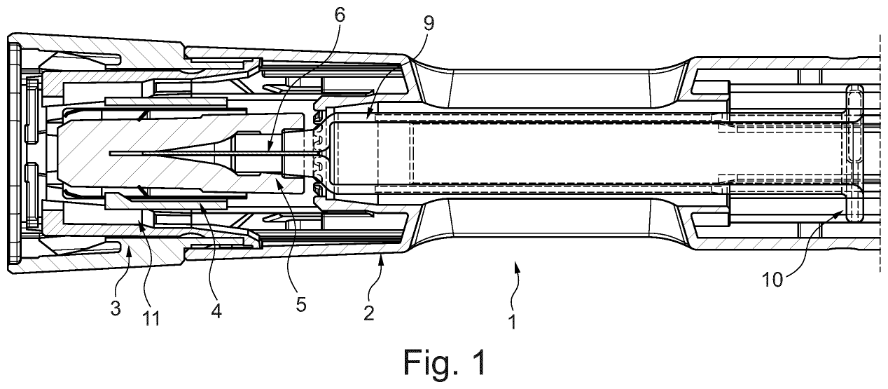

FIG. 1 shows a drug delivery device 1 in the form of an injection pen according to an embodiment. The drug delivery device 1 may be an autoinjector. The drug delivery device 1 may be a disposable device. In addition, the device 1 may be a fixed dose device capable of delivering multiple individual predetermined doses. The drug delivery device 1 may have an elongated shape relative to the bearing surface on the body of the user. The device 1 has a distal end (left end in FIG. 1) and a proximal end (right end in FIG. 1). The term “distal” refers to a location that is relatively closer to a site of injection, and the term “proximal” refers to a location that is relatively further away from the injection site. The device 1 includes a housing 2 and a cap 3, which covers the distal end of the housing 2, when connected to the housing 2. The cap 3 includes a needle shield remover 4 configured to be rotationally locked to a needle shield 5 covering a needle 6. The needle shield remover 4 is connected to the needle shield 5 when the cap 3 is connected to the housing 2, e.g. by a positive connection. The needle shield remover 4 is removed together with the cap 3, when the cap 3 is removed from the housing 2. Furthermore, the needle shield remover 4 is received in a sleeve-like portion of the cap 3. The needle shield remover 4 is rotationally and axially locked relative to the cap 3. The arrangement further comprises a plunger rod, a drive spring holder (or housing part) and an energy storage member (not shown) having energy stored therein, which energy, when released, drives the plunger rod in the distal direction relative to the housing 2. The energy storage member may be a drive spring.

Moreover, the device 1 comprises a needle shroud 11, the needle shroud 11 being axially movable relative to the housing 2. The dispensing of the drug is triggered by an axial movement of the needle shroud 11 relative to the housing 2.

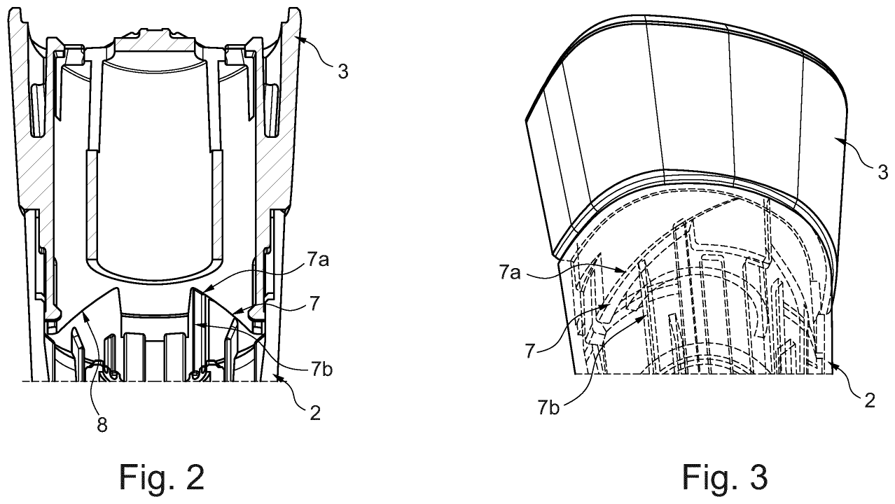

As shown in FIG. 2, the needle shield remover 4 is removably connected to the housing 2, wherein the drug delivery device 1 comprises a guiding interface 7. The guiding interface 7 is formed between the cap 3 and the housing 2. The guiding interface 7 comprises a guide track 7a and an interaction feature 7b. The guiding interface 7 is configured such that when the needle shield remover 4 rotates with respect to the housing 2 from a state in which the needle shield remover 4 is connected to the housing 2, the guiding interface 7 guides movement of the needle shield remover 4 relative to the housing 2 in a direction away from the proximal end of the housing 2 to disconnect the needle shield remover 4 together with the needle shield 5 from the housing. The movement of the needle shield remover 4 to disconnect the needle shield remover 4 from the housing 2 comprises axial and rotational movement of the needle shield remover 4 relative to the housing 2. Moreover, the movement of the needle shield remover 4 relative to the housing 4 comprises a simultaneous axial and rotational movement of the needle shield remover 4 relative to the housing 2.

However, it is also possible that the movement of the needle shield remover 4 relative to the housing 2 does not comprises a simultaneous axial and rotational movement. In this case, it may be possible that a rotational movement takes place at first and the axial movement only takes place after the rotary movement has been completed, or vice versa. Furthermore, it is conceivable that the cap 3 must first be moved relative to the housing 2 only axially before the cap 3 can subsequently be rotated relative to the housing 2.

In the present embodiment the needle shield remover 4 is part of a cap assembly which comprises the cap 3 and the needle shield remover 4, wherein, in the cap assembly, the needle shield remover 4 is assembled to the cap 3, and wherein, in the cap assembly, the needle shield remover 4 is rotationally locked to the cap 3, e.g. with respect to an exterior surface of the cap 3. The exterior surface is provided as an interface surface for being touched by a user when removing the cap 3 from the housing 2. Therefore, a rotation of the cap 3 directly leads to a rotation of the needle shield remover 4.

As shown in FIG. 2, the arrangement comprise a further guiding interface 8 which is configured to guide movement of the needle shield remover 4 relative to the housing 2 during disconnection of the needle shield remover 4 from the housing 2. The rotational direction defined by the further guiding interface 8 is opposite to the one defined by the guiding interface 7. It should be noted that the further guiding interface 8 may also have all the properties of the guiding interface 7 mentioned above and below.

The guiding interface 7 defines a rotation direction for the rotation required to disconnect the needle shield remover 4 and thus the needle shield 5 from the housing 2. The guiding interface 7 is configured such that rotation in only one direction is permitted to disconnect the needle shield remover 4 from the housing 2. As shown in FIGS. 2 and 3 the guiding interface 7 is established between the cap 3 and the housing 2 and comprises at least one guide track 7a, which is arranged on a part of the cap 3, and at least one interaction feature 7b, which is arranged on a part of the distal end of the housing 2. In the shown embodiment the interaction feature 7b is a rib and the end geometry of the rib matches that of the guide track 7a. Accordingly, the end geometry of the rib and the geometry of the guide track 7a are complementary in shape. In the shown embodiment, the rib is located on the inside of the housing 2. As the cap 3 is rotated, the rib lifts the cap 3 off the housing 2 due to a direct engagement of the rib with the guide track 7a. The guide track 7a is configured to guide the movement of the needle shield remover 4 relative to the housing 2 when the needle shield remover 4 is disconnected from the housing 2. The guide track 7a has at least one sloped region. However, it is also possible that the guide track 7 comprises a first sloped region and a second sloped region, wherein the first sloped region and the second sloped region have different slopes to each other and thus lead to different axial displacement of the cap 3 relative to the housing 2 during the removal of the cap 3.

In the second sloped region, on account of a smaller axial displacement, more force is available inside the needle shield remover 4, e.g. to move the needle shield 5 together with needle shield remover 4, than in the first sloped region. Thus, the guide track 7 can be adjusted to the needs during a cap removal process. In an initial phase, where the first sloped region guides the movement an internal force in the housing 2, e.g. a frictional force, counteracting the cap removal movement may be less than in the second sloped region. Thus, advantageously, the first sloped region may be steeper and/or require less space in the arrangement. When the higher force is required, however, the slope may be reduced to increase the proportion of the user force which is transferred to the needle shield remover 4. Having sloped regions in the guiding interface 7 when removing the cap 3 provides a mechanical advantage as compared to a situation where the user tries to axially pull of the cap 3. As during cap removal high forces have to be exerted having an improved transmission to reduce the force which the user has to generate to remove the cap 3 is advantageous. The force which has to be exerted while removing the cap 3 by a pure axial pulling movement is greater than or equal to one of the following values: 15N, 20N, 25N, 30N, 31N, 32N, 33N, 34N, 35N.

The minimum force required to move the needle shield 5 may not be the highest initially. Rather there may be an increasing force required during the movement. Several gradients within the guide track 7a may tailor the guiding interface 7 to the specific application where an increased force is transferred via the needle shield remover 4 after an initial movement has been performed already. As the force counteracting the removal of the cap 3 is initially smaller than later on, but a force maximum occurs close to the beginning, it is expedient to have a first sloped region with an extension less than the second sloped region, wherein the force maximum, either a local or global maximum during the entire cap removal travel, expediently occurs while the second sloped region guides the movement of the needle shield remover 4 relative to the housing 2.

The first sloped region may be steeper than the second sloped region. In other words, a slope in the first slope region may be greater than a slope in the second sloped region. The slope in the respective region may be constant in this case. A steep (great) slope of the guide track 7 is advantageous in regions where the force required to move the needle shield 5 is small, e.g. smaller than in other phases during the cap removal process. It is therefore possible to choose the slope of the guide track 7a depending on the force required to remove the needle shield 5. Thus, the embodiment is in particular advantageous in cases where the required force increases during the removal of the needle shield 5. Using steeper slopes in areas whose traverse requires little force is further advantageous because the steeper slope results in greater axial movement of the cap 3 at a given angle of rotation. When the force required to move the needle shield remover 4 or keep the needle shield remover 4 moving increases, the slope can be reduced in the associated phase of the cap removal or detaching process. Thus, the cap 3 is removed faster and/or the space consumption of the guide track 7a or the guiding interface 7 is comparatively small.

In case of two sloped regions a pure axial extension of the first sloped region may be smaller or greater than or equal to a pure axial extension of the second sloped region, e.g. as seen along the rotation axis. In this context, the term “pure axial extension” means the length of the respective sloped region measured along the longitudinal axis of the arrangement or the housing or the rotation axis.

The slope of the guide track 7 in the first sloped region and/or the second sloped region may be constant. For example, the slope of the first and second sloped region may have a value between 15° and 75°, e.g. 45°.

A ramp angle defining the slope of the first sloped region may be greater than a ramp angle defining the slope of the second sloped region. The respective ramp angle may be measured relative to the rotation axis or the longitudinal axis of the housing or the assembly. The ramp angle defining the slope of the first sloped region may be greater than or equal to 30° and/or less than or equal to 60°. A ramp angle of more than 30° and less than 60° provides considerable axial displacement of the cap 3 and a sufficient transmission ratio while the cap 3 is traversing the first sloped region of the guide track 7a. In particular, a ramp angle of 40° for the first sloped region is advantageous.

The ramp angle defining the slope of the second region may be greater than or equal to 10° and/or less than or equal to 30°. A ramp angle of more than 10° and less than 30° provides a sufficient axial displacement of the needle shield remover 4 and an optimized transmission ratio, to increase the proportion of the user force transferred, while the cap 3 is traversing the second sloped region of the guide track 7a. In particular, a ramp angle of 25° for the second sloped region is advantageous. A maximum of force which has to be exerted during the removal process to move the needle shield remover 4 or keep the needle shield remover 5 moving, may fall within the range of movement defined by the second sloped region during removal of the cap 3 from the housing 4. It is therefore advantageous if the second sloped region has a smaller slope to obtain a larger gear ratio.

The slope of the first sloped region and/or the second sloped region may vary. The slope of the first sloped region and second sloped region does not have to be constant. If the slope varies within one of the regions, it is preferred that, in a first section closer to the start of the sloped region, the slope is smaller than in a subsequent section closer to the end of the sloped region.

Furthermore, the first sloped region may be directly connected to the second sloped region. The axial movement of the cap 3 away from the housing 2 is thus not interrupted during a rotational movement of the cap 3.

It is also possible, that the guide track 7a has a third sloped region. When the arrangement is switched from the capped state to the uncapped state, the third sloped region guides the movement of the cap 3 after the second sloped region. The first sloped region, the second sloped region and the third sloped region may be adjusted to one another such that, when the third sloped region guides the movement, and the cap 3 is rotated, the cap 3 is displaced axially away from the housing 2 by a third distance. The second distance may be smaller than the third distance. The first distance may be greater than, less than, or equal to the third distance. The addition of a third sloped region further facilitates removal of the cap 3 as the third sloped region may provide an increased transmission, after the maximum force(s) which have to be exerted for rotating the needle shield remover 4 have occurred already during the cap removal process. It should be noted that the guide track 7a may also comprise more than three sloped regions. However, three sloped regions, where the intermediate region, here the second sloped region, is adjusted for an optimal force transfer from the cap to the needle shield may be advantageous nevertheless in terms of space consumption.

The third sloped region may be steeper than the second sloped region. As mentioned above a greater slope on the guide track 7a is advantageous in regions where the force required to remove the cap 3 is smaller, e.g. on account of the smaller space consumption or footprint required in the assembly. It is therefore possible to choose the slope of the guide track 7a depending on the force required to move the needle shield remover 4. Thus, such an embodiment is in particular advantageous in cases where the required force decreases after traversing the second sloped region. Using steeper slopes in areas which require comparatively little force to traverse is further advantageous because the steeper slope results in greater axial displacement of the cap 3 at a given rotation angle. Therefore, the cap 3 is removed faster. Moreover, the user may have an experience similar to the one perceived while traversing the first sloped region, which increases the user's confidence that the cap 3 removal process works as it should.

The third sloped region may be as steep as the first sloped region. Accordingly, the first and third regions may have equal slopes. This is advantageous if the force to be overcome during the traversing of the first and third sloped regions is lower, since for these areas a more pronounced axial displacement of the needle shield remover 4 is achieved for a given rotation angle.

The pure axial extension of the third sloped region may be less or greater than, or equal to the pure axial extension of the second sloped region. As mentioned above, in this context, the term “pure axial extension” stands for the length of one of the sloped regions measured parallel along the longitudinal axis of the arrangement. In other words, the pure axial extension is equal to the axial offset between the opposite ends of the region.

The pure axial extension of the first sloped region may be in the range of 0.1 to 1 mm, the pure axial extension of the second sloped region may be in the range of 4 to 6 mm, and/or the pure axial extension of the third sloped region may be in the range of 0.5 to 2 mm.

The pure axial extension of all of the sloped regions when taken together is greater than or equal to 5 mm and/or less than or equal to 8 mm. This is advantageous because the axial distance to remove the cap 3 is short, resulting in quick removal of the cap 3.

The slope of the third sloped region may be constant.

A ramp angle defining the slope of the third sloped region may be greater than the ramp angle defining the slope of the second region.

A ramp angle defining the slope of the third region may be greater than 30° and/or less than 60°. A ramp angle of more than 30° and less than 60° provides considerable axial displacement of the needle shield remover 4 and a sufficient transmission ratio while the cap 3 is traversing the third sloped region of the guide track 7a. In particular, a ramp angle of 40° for the third sloped region is advantageous.

The ramp angle of the third sloped region may be equal to the ramp angle of the first sloped region. This is advantageous if the force to be overcome during the traversing of the first and third sloped regions is low, since for these areas a rapid axial feed of the cap 3 is achieved.

The slope of the third sloped region may vary. The slope of the third sloped region does not have to be constant. The third sloped region may be directly connected to the second sloped region. The first sloped region may be directly connected to the second sloped region and the second sloped region may be directly connected to the third sloped region.

As shown in FIG. 1, the housing 2 has a reservoir 9 filled with drug. The reservoir 9 comprises a reservoir body with a discharge opening, an interior which is configured to retain a medicament, and the needle 6 for providing fluid communication between the interior and the exterior of the reservoir body. The reservoir body may be of glass. The needle 6 is covered by the needle shield 5. The needle shield 5 is removably connected to the reservoir 9, covering the discharge opening of the reservoir 9, e.g. the tip of the needle 6. The reservoir 9 may also be a pre-filled syringe. When the needle shield remover 4 is disconnected from the housing 2, the reservoir rotates together with the needle shield remover 4 relative to the housing 2 from an initial reservoir position relative to the housing 2 to an end reservoir position relative to the housing 2.

The needle shield remover 4 is connected to the needle shield 5 such that the needle shield remover 4 removes the needle shield 5 from the reservoir 9, when the cap 3 is removed from the housing 2. The force required to move the needle shield 5 in an axial direction relative to the reservoir 9, e.g. along the reservoir and/or away from the needle 6 may vary.

The force may have a maximum which occurs while the needle shield 5 is removed from the reservoir 9. The guide track 7a may be configured such that the maximum falls within the range of movement defined by the second sloped region during removal of the cap 3 from the housing 2. This is advantageous if the second sloped region comprises the largest transmission ratio of guide track 7a.

The needle shield 5 comprises a rigid outer area and an elastic inner area, wherein the rigid outer area faces the needle shield remover 4 and the elastic inner area faces the discharge opening of the reservoir 9. The needle shield is a so-called rigid needle shield, which has a rigid outer shell and a deformable inner part which contacts the needle 6. The force required to pull off the needle shield 5 from the reservoir 9 may be greater than 30 N.

While the rigid outer area allows or facilitates a positive connection with the needle shield remover 4, the elastic inner area enables a frictional and/or tight connection with the reservoir 9 and/or the needle 6.

In the embodiment shown here, the reservoir 9 is at least partially rotatable relative to the housing 2. Accordingly, the reservoir 9 can at least partially follow the rotational movement of the needle shield remover 4 when the cap 3 is removed. This has the advantage that an undesired twisting of the needle shield when the cap is removed can be counteracted. As shown in FIG. 1, the reservoir 9 comprises a reservoir flange 10, which is located at the proximal end of the reservoir 9. Restricted rotational mobility of the reservoir 9 can result from the geometry of the reservoir flange 10.

The reservoir flange 10 can be rotationally symmetrical in the axial direction, e.g. for arbitrary rotation angles or just one rotation angle, such as 180°. However, it is also possible that the reservoir flange 10 is not rotationally symmetrical. In the latter case, the shape of the reservoir flange 10 can restrict its rotatability and thus the rotatability of the reservoir 9 with respect to the housing 2 if the rotation of the reservoir flange 10 is limited by a rotation stop in the housing 2, the rotation stop being rotationally fixed relative to the housing 2. Thus, during removal of the cap 3, a first angle, which is a rotation angle of the reservoir 9 between an initial reservoir position and an end reservoir position may be less than a second angle, which is a rotation angle of the needle shield remover 4 between an initial needle shield remover position and an end needle shield remover position, particularly relative to the housing 2. In other words, in this case the reservoir 9 can only partially follow the rotation of the needle shield remover 4. It may be sufficient if the reservoir 9 does not take part in the entire rotational movement of the needle shield remover 4, but only during a part of the rotational movement, e.g. that part which requires the greatest force. Thus, if the cap 3 and the needle shield remover 4 are rotated while removing the cap 3 from the housing 2, the reservoir 9 and the needle shield remover 4 will rotate together at least until a maximum force to remove the needle shield 5 is overcome.

The first angle may be greater than or equal to 15°, 30°, 45°, 60°, 75°, 90°, 105°, 120°, 135°, 150°, 165°, 180°, 195°, 210°, 225°, 240°, 255°, 270°, 285°, 300°, 315°, 330°, 345° or 360°, wherein the second angle may greater than or equal to 15°, 30°, 45°, 60°, 75°, 90°, 105°, 120°, 135°, 150°, 165°, 180°, 195°, 210°, 225°, 240°, 255°, 270°, 285°, 300°, 315°, 330°, 345° or 360°.

The first angle may be less than or equal to 15°, 30°, 45°, 60°, 75°, 90°, 105°, 120°, 135°, 150°, 165°, 180°, 195°, 210°, 225°, 240°, 255°, 270°, 285°, 300°, 315°, 330°, 345° or 360°, wherein the second angle may be less than or equal to 15°, 30°, 45°, 60°, 75°, 90°, 105°, 120°, 135°, 150°, 165°, 180°, 195°, 210°, 225°, 240°, 255°, 270°, 285°, 300°, 315°, 330°, 345° or 360°.

In the shown example the first angle may be between 15° and 90° and the second angle may between 30° and 180°. The first angle may be between 15° and 90°. The second angle may be between 30° and 180°. Both, the first angle and the second angle may be 15°, 30°, 45°, 60°, 75°, 90°, 105°, 120°, 135°, 150°, 165° or 180°.

The needle shroud 11 preferably has no protruding portions (for example guide ribs) for guiding the plunger that may be provided to restrict the rotation of the reservoir 9 relative to the needle shroud 11. In the shown embodiment, the functionality of protruding portions like guide ribs may be replaced by lengthened ribs on the drive spring holder or housing part. Thus, guide functionality for guiding the plunger may be performed by the drive spring holder or housing portion. In this way, rotatability of the reservoir 9 relative to the needle shroud 11 may not be impeded by structure, e.g. ribs, on the needle shroud 11.

In order to ensure stable positioning of the reservoir 9 in the housing 2, the reservoir 9 is fixed in the axial direction relative to the housing 2, at least after the cap 3 has been removed, that is to say immediately before the medication is dispensed or the dispensing is triggered. Therefore, in the present embodiment, the reservoir 9 is fixed in the axial direction relative to the housing 2, at least after the cap 3 has been removed. This may be assisted or achieved by a biasing force of a reservoir biasing system which is applied via the reservoir flange 10 to the reservoir 9 in the axial direction relative to the distal end of the housing 2, at least after the cap 3 has been removed. It should be noted that a pretension or bias can also exist in the exemplary embodiment shown before the cap 3 is removed. It is also possible that this force varies during the removal of the cap 3.

The reservoir biasing system may comprise the reservoir flange 10, the drive spring holder and a biasing means, e.g. an elastically deformable portion of the drive spring holder, wherein the biasing means is located between the reservoir flange 10 and the drive spring holder. The biasing means biases the reservoir 9 with a force in the distal direction relative to the housing 2, preferably during removal of the cap 3 from the housing 2. The force applied by of the biasing means towards the reservoir flange 10 may be constant or variable throughout the removal of the cap 3. In cases where the force is variable, the force applied by the biasing means may increase throughout the removal of the cap 3. However it is also conceivable that the force applied by the biasing means decreases throughout the removal of the cap 3. A decrease or increase in the force can be achieved, for example, by the drive spring holder having ramps which compress the biasing means as a function of the rotation of the reservoir flange 10 relative to the housing 2, either less or more for decreasing or increasing the bias. The biasing system may ensure that the reservoir is reliably positioned within the housing 2, e.g. taking into account tolerances between different reservoirs.

The delivery of a drug using a drug delivery device having the arrangement disclosed herein is as described below.

A user first grips the outer peripheral surface of the cap 3. He then turns the cap 3 in one direction relative to the housing 2. By rotating the cap 3 relative to the housing 2, the guide track 7a of the cap 3 is moved along the complementary shape of the interaction feature 7b of, i.e. the end of the rib, which is part of the housing 2. Due to the slope of the guide track 7a the cap 3 is displaced in the axial direction relative to the housing 2 and the cap 3 is thus increasingly removed from the housing 2. Since the needle shield remover 4 is rotational locked relative to the cap 3, a rotation of the cap 3 immediately leads to a rotation of the needle shield remover 4. As the needle shield remover 4 rotates when being disconnected from the housing 2 and the needle shield remover 4 is rotationally and axially locked to the needle shield 5, the force driving the rotation is used to pull the needle shield 5 from the needle 6. Since the reservoir 9 is rotatably supported at least in sections relative to the housing 2, the reservoir 9 together with the needle shield 5 can follow the rotation of the cap 3 and thus also the rotation of the needle shield remover 4 when the cap 3 is removed from the housing 2. This ensures that the needle shield 5 is primarily subjected to a force in the axial direction during removal although the needle shield remover 4 is rotationally locked to the needle shield 5. In this way, an undesired twisting of the needle shield 5 during its removal can be avoided. After the cap 3 is removed the user presses the device 1 against the skin, inserting the needle and depressing the needle shroud 11. Depression of the needle shroud allows the plunger to commence the injection.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a reservoir, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber reservoir configured to store two or more components of the pharmaceutical formulation to-be-administered (e.g., an API and a diluent, or two different drugs) separately, one in each chamber. In such instances, the two chambers of the dual-chamber reservoir may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

### REFERENCE NUMERALS

1 drug delivery device
2 housing
3 cap
4 needle shield remover
5 needle shield
6 needle
7 guiding interface
7a guide track
7b interaction feature
8 further guiding interface
9 reservoir
10 reservoir flange
11 needle shroud

1-16. (canceled)

17. An arrangement for a drug delivery device, the arrangement comprising:
a housing with a proximal end and a distal end;
a needle shield remover configured to be rotationally locked to a needle shield covering a needle, wherein the needle shield remover is releasably connected or releasably connectable to the housing; and
a guiding interface,
wherein the guiding interface is configured such that, when the needle shield remover rotates with respect to the housing from a state in which the needle shield remover is connected to the housing, the guiding interface guides a movement of the needle shield remover relative to the housing in a direction away from the proximal end of the housing to disconnect the needle shield remover from the housing, and
wherein the movement of the needle shield remover relative to the housing to disconnect the needle shield remover from the housing comprises an axial movement and a rotational movement of the needle shield remover relative to the housing.

18. The arrangement according to claim 17, wherein the axial and rotational movements of the needle shield remover relative to the housing occur simultaneously.

19. The arrangement according to claim 17, wherein the needle shield remover is a cap or comprises a part of a cap assembly

20. The arrangement according to claim 19, wherein, in the cap assembly, the needle shield remover is assembled with a cap and rotationally locked to the cap.

21. The arrangement according to claim 17, wherein the guiding interface defines a rotational direction for a rotation required to disconnect the needle shield remover from the housing.

22. The arrangement according to claim 17, wherein the guiding interface is configured such that a rotation in only one direction is permitted to disconnect the needle shield remover from the housing.

23. The arrangement according to claim 17, wherein the guiding interface comprises at least one guide track that is configured to guide the movement of the needle shield remover relative to the housing when the needle shield remover is disconnected from the housing.

24. The arrangement according to claim 17, wherein the guiding interface is a first guiding interface, and wherein the movement is a first movement,
wherein the arrangement further comprises a second guiding interface that is configured to guide a second movement of the needle shield remover relative to the housing during a disconnection of the needle shield remover from the housing, and wherein a second rotational direction defined by the second guiding interface is opposite to a first rotational direction defined by the first guiding interface.

25. The arrangement according to claim 17, wherein the housing is configured to receive a reservoir or the reservoir is received in the housing, wherein the reservoir comprises a reservoir body with an interior region that is configured to retain a medicament and the needle for providing a fluid communication between the interior region of the reservoir body and an exterior portion of the reservoir body, and wherein the needle is covered by the needle shield.

26. The arrangement according to claim 25, wherein the reservoir, when received in the housing, is axially secured relative to the housing, and wherein an axial position of the reservoir relative to the housing does not change when the needle shield remover is being disconnected from the housing.

27. The arrangement according to claim 25, wherein the reservoir is configured such that, when the needle shield remover is being disconnected from the housing, the reservoir can rotate together with the needle shield remover relative to the housing from an initial reservoir position relative to the housing to an end reservoir position relative to the housing.

28. The arrangement according to claim 25, wherein the needle shield remover is configured such that, when the needle shield remover is disconnected from the housing, the needle shield remover rotates from an initial needle shield remover position relative to the housing to an end needle shield remover position relative to the housing

29. The arrangement according to claim 28, wherein a rotation angle of the reservoir between the initial reservoir position and the end reservoir position is a first angle, wherein a rotation angle of the needle shield remover between the initial needle shield remover position and the end needle shield remover position is a second angle, and wherein the first angle is equal to the second angle.

30. The arrangement according to claim 27, wherein the needle shield remover is configured such that, when the needle shield remover is disconnected from the housing, the needle shield remover rotates from an initial needle shield remover position relative to the housing to an end needle shield remover position relative to the housing

31. The arrangement according to claim 30, wherein a rotation angle of the reservoir between the initial reservoir position and the end reservoir position is a first angle, wherein a rotation angle of the needle shield remover between the initial needle shield remover position and the end needle shield remover position is a second angle, and wherein the first angle is less than the second angle.

32. The arrangement according to claim 17, further comprising:
a plunger rod; and
an energy storage member having energy stored therein, wherein the energy, when released, drives the plunger rod in a distal direction relative to the housing.

33. A drug delivery device comprising an arrangement, the arrangement comprising:
a housing with a proximal end and a distal end;
a needle shield remover configured to be rotationally locked to a needle shield covering a needle, wherein the needle shield remover is releasably connected or releasably connectable to the housing; and
a guiding interface,
wherein the guiding interface is configured such that, when the needle shield remover rotates with respect to the housing from a state in which the needle shield remover is connected to the housing, the guiding interface guides a movement of the needle shield remover relative to the housing in a direction away from the proximal end of the housing to disconnect the needle shield remover from the housing, and
wherein the movement of the needle shield remover relative to the housing to disconnect the needle shield remover from the housing comprises an axial movement and a rotational movement of the needle shield remover relative to the housing.

34. The drug delivery device according to claim 33, wherein the drug delivery device comprises an autoinjector.

35. The drug delivery device according to claim 34, further comprising a reservoir filled with a drug.

36. An arrangement for a drug delivery device, the arrangement comprising:
a housing with a proximal end and a distal end;
a needle shield remover configured to be rotationally locked to a needle shield covering a needle, wherein the needle shield remover is releasably connected or releasably connectable to the housing; and
a guiding interface,
wherein the guiding interface is configured such that, when the needle shield remover rotates with respect to the housing from a state in which the needle shield remover is connected to the housing, the guiding interface guides a movement of the needle shield remover relative to the housing in a direction away from the proximal end of the housing to disconnect the needle shield remover from the housing,
wherein the movement of the needle shield remover relative to the housing to disconnect the needle shield remover from the housing comprises an axial movement and a rotational movement of the needle shield remover relative to the housing,
wherein the housing is configured to receive a reservoir or the reservoir is received in the housing, the reservoir comprising a reservoir body with an interior region that is configured to retain a medicament and the needle for providing a fluid communication between the interior region of the reservoir body and an exterior portion of the reservoir body,
wherein the needle is covered by the needle shield, and
wherein the reservoir is configured such that, when the needle shield remover is being disconnected from the housing, the reservoir can rotate together with the needle shield remover relative to the housing from an initial reservoir position relative to the housing to an end reservoir position relative to the housing.
